(19) United States
(12) Patent Application Publication
(10) Pub. No.: US 2025/0102499 A1
(43) Pub. Date: Mar. 27, 2025

(54) SYSTEMS AND METHODS FOR DISEASE DETECTION

(71) Applicant: Mercy Bioanalytics, Inc., Waltham, MA (US)

(72) Inventors: Toumy Guettouche, Waltham, MA (US); Laura Teresa Bortolin, Waltham, MA (US); Dawn Mattoon, Waltham, MA (US); Lauren Taylor Cuoco, Waltham, MA (US)

(21) Appl. No.: 18/889,935

(22) Filed: Sep. 19, 2024

Related U.S. Application Data
(60) Provisional application No. 63/584,627, filed on Sep. 22, 2023.

(51) Int. Cl. G01N 33/543

(52) U.S. Cl. CPC G01N 33/54326

(57) ABSTRACT

The invention provides methods and kits for the analysis of extracellular vesicle-derived biomarkers obtained from dried body fluid samples; including but not limited to blood spots extracted onto or into absorptive surfaces for analysis of extracellular vesicles for disease diagnosis, staging, monitoring and selection and efficacy of therapeutics.

### TECHNICAL FIELD

The invention relates to devices and methods for sample collection and testing.

### BACKGROUND

Proper sample collection is critical to the provision of quality healthcare. Many conventional diagnostic tests are conducting on venous blood samples. However, those conventional approaches have drawbacks. Some people may be reluctant or unable to give blood at a collection center. For instance, many individuals in rural areas and the elderly often have difficulty getting to a clinical laboratory. Also, anxiety around venipuncture contributes to low compliance. Thus, the presentation at a clinic may contribute to disengagement from needed medical care. There is, therefore, a need for techniques that improve access to diagnostic testing.

### SUMMARY

The invention provides methods and devices for sample collection for use at home or in the field. Devices and methods of the invention are useful for the collection of any bodily fluid sample. According to the invention, a bodily fluid is spotted onto an absorptive matrix and dried, after which the matrix (or device housing the matrix) is shipped to a laboratory for analysis. In one aspect, a clinical laboratory isolates extracellular vesicles from the dried spot on the collection device for analysis. Extracellular vesicles provide specific disease biomarkers associated with tissue, including, for example, tumor cells. Because tumor cells release extracellular vesicles into circulation, bodily fluids, such as blood, can be probed to detect the presence of cancer. According to the invention, extracellular vesicles can be preserved intact within, and later recovered from, a dried blood spot or other similar sample. It is a feature of the invention, that dried bodily fluid samples (e.g., blood) can be sent via the mail or a courier service to a reference laboratory. Thus, the invention provides detection kits with instructions to collect one or more samples on a collection card, which is then sent to the laboratory. In a preferred embodiment, the collection card comprises a matrix on which the bodily fluid is deposited and that promotes retention and elution of extracellular vesicles.

Another feature of the invention is that samples can be collected multiple times over a defined period in order to increase the likelihood of obtaining a representative sample (e.g., after fasting or at certain times during the day). Because sample collection can be performed at home, certain geographical, personal, or financial obstacles are minimized.

In certain aspects, the invention provides analysis methods that include obtaining a sample collection device having a dried bodily fluid sample (e.g., blood) on a surface thereof, extracting at least one extracellular vesicle from the dried bodily fluid sample, detecting at least one biomarker associated with the extracellular vesicles, and determining health status of the patient based upon the biomarker(s). In a preferred embodiment, the co-occurrence of at least three biomarkers on the extracellular vesicle is used to determine health status. The three biomarkers are preferably selected based on their co-occurrence in cells indicative of the medical condition to be diagnosed.

The bodily fluid used in methods of the invention may be obtained, for example, by finger prick and wicking the fluid onto an absorptive matrix (e.g., cellulose). The collection device comprises a matrix that receives the sample. The sample is dried onto the matrix within the device. The matrix may be pre-treated with a sugar, an excipient, a surfactant, an antimicrobial agent, an antifungal agent, a proteinase inhibitor, a DNase inhibitor, an anticoagulant, and/or a desiccant. Methods and devices of the invention may also include a mailer and instructions for returning the obtained sample to a reference laboratory.

In some embodiments, extracellular vesicles are extracted from the dried sample spot by elution into an aqueous mixture. Extracellular vesicles are analyzed by detecting the presence of disease-related biomarkers. In a preferred embodiment, the biomarkers are present on the surface of the vesicles. In a particular assay, disease diagnosis is accomplished by analyzing multiple coincident biomarkers on the vesicles using, for example, using a proximity ligation assay. For example, an assay may include binding two oligos to each of two biomarkers, ligating the two oligos into a single nucleic acid construct, and detecting the nucleic acid construct. Preferably, the proximity ligation assay uses two antibody-DNA conjugates. The DNA portion of the two antibody-DNA conjugates may be ligated together and detected by a reaction, such as quantitative PCR.

Certain aspects of the invention provide sample collection devices. Preferred sample collection devices include a substrate (such as cardstock or plastic), at least one absorptive matrix on a surface of the substate, a lancet, and a sterilizing agent (e.g., ethyl alcohol). The device may include markings on the surface showing a user where to place a bodily fluid sample and at least one capillary structure to draw the bodily fluid sample into the absorptive matrix. The absorptive matrix may be pre-treated with a sugar, an excipient, a surfactant, an antimicrobial agent, an antifungal agent, a proteinase inhibitor, a DNase inhibitor, an anticoagulant, or a desiccant. The absorptive matrix may be filter paper, cellulose, paper, or some other fibrous material. Preferably, the absorptive matrix is soluble in aqueous solutions. In some embodiments, the absorptive matrix has an open internal structure that promotes absorption of fluid by capillary action and internal spaces dimensioned to receive released extracellular vesicles and hold the extracellular vesicle intact. The sterilizing agent may be ethyl alcohol (e.g., allowing a user to wipe and disinfect a finger to provide a sample that is not contaminated). The device may be be shipped to a testing facility through a mail carrier (e.g., and may be pre-printed with shipping information). In a preferred embodiment, the absorptive matrix contains reagents for the elution and isolation of extracellular vesicles.

In multi-spot embodiments, the surface of the substate has at least (i) a first sample collection location that include the absorptive matrix and (ii) a second sample collection location that includes a second absorptive matrix. The device may come with a package that includes a lancet, a return envelope, and instructions directing the collection of multiple samples.

Any type of body fluid may be used in accordance with the invention such as blood, sputum, urine. In a preferred embodiment, the body fluid is blood. Blood is obtained via a finger prick using a capillary or a lancet. In one aspect, the blood is spotted onto the absorptive matrix and air-dried. The absorptive matrix may be a filter paper, cellulose, paper or any similar absorptive material.

Aspects of the invention involve providing to a user an at-home collection device for collection of the body fluid sample. A preferred collection device comprises an absorptive material, a sterilizing agent, a capillary or a lancet for blood collection, and packaging for returning the collection device to a laboratory for further analysis. Once the user collects a blood spot on the absorptive matrix, air-dries the spot, and returns it to the laboratory, dried blood spots are analyzed in the laboratory for presence of disease-specific biomarkers.

In a preferred embodiment, extracellular vesicles are eluted from the dried blood spot for detection of disease. In preferred embodiments, the extracellular vesicles are isolated from the dried blood spot using size exclusion. The isolated extracellular vesicles are assayed for the presence of disease-related biomarkers. The invention utilizes any molecular biology technique for identification, characterization, analysis and/or quantification of disease related biomarkers on the surface of the extracellular vesicles. Such techniques include, but are not limited to, PCR, antibody-DNA conjugates, detection probes. In one non-limiting embodiment, extracellular vesicles having co-localization of at least three of more surface biomarkers are considered as target extracellular vesicles. Target extracellular vesicles are contacted with a capture agent directed to an extracellular vesicle associated surface biomarker. In some embodiments, such a capture agent may comprise a binding moiety directed to an extracellular vesicle-associated surface, which may be optionally conjugated to a solid substrate. An exemplary capture agent for an extracellular vesicle-associated membrane-bound polypeptide may comprise a solid substrate (e.g., a magnetic bead) and a binding moiety (e.g., an antibody agent) directed to an extracellular vesicle-associated membrane-bound polypeptide. Captured extracellular vesicles are incubated with a set of antibody-DNA conjugates having a double-stranded DNA comprising a complementary single-stranded overhang. When the antibody-DNA conjugates are bound to two biomarkers that co-occur on the same extracellular vesicle, the single stranded overhang portion of the antibody-DNA conjugates hybridize to each other. Such a combination comprising the extracellular vesicles and the set of detection probes is then maintained under conditions that permit binding of the set of detection probes to their respective targets on the extracellular vesicles such that their oligonucleotide domains are in close enough proximity to anneal to form a double-stranded complex. Such a double-stranded complex can be detected by contacting the double-stranded complex with a nucleic acid ligase to generate a ligated template; and detecting the ligated template. In some embodiments, a ligated template can be detected using quantitative PCR. The presence of such a ligated template is indicative of presence of extracellular vesicles that are positive for a target biomarker signature of disease.

The invention is useful for detection of any type of disease but is especially applicable to diagnosis and screening of cancer. The invention also contemplates methods for analysis of disease progression, including severity, staging, and recurrence (including minimal residual disease). It is known, for example, that changes (increasing or decreasing amounts, structural alterations, sequence changes) in certain biomarkers are associated with specific diseases.

Accordingly, methods of the invention track biomarkers in extracellular vesicles for changes indicative of, for example, disease severity or stage.

### BRIEF DESCRIPTION OF THE DRAWINGS

FIG. 1 shows a sample collection device.

FIG. 2 shows extracellular vesicles in an aqueous suspension a tube.

FIG. 3 illustrates steps of a proximity ligation assay.

FIG. 4 shows steps of an analysis method of the invention.

### DETAILED DESCRIPTION

The present invention generally relates to methods and devices for sample collection and detection of disease. In particular, the invention provides methods and devices for detecting disease by analyzing extracellular vesicles for presence of disease-specific biomarkers from dried bodily fluid.

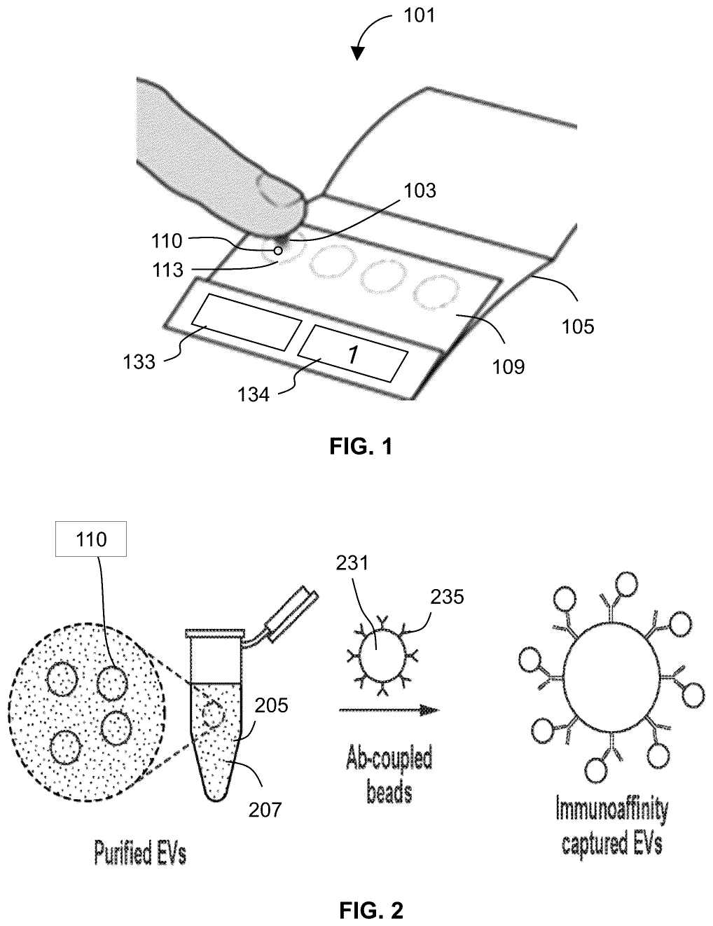

FIG. 1 shows a sample collection device 101 for collecting a bodily fluid sample 103. Exemplary sample collection devices include a substrate 109 with an absorptive matrix 113 on its surface. The device 101 may include a reagent 133, and optionally additional reagents 134, e.g., pre-loaded on the substrate 109 or an enclosed wrapper or package 105. The example, the reagent 133 may be a sterilizing agent (e.g., ethyl alcohol). The additional reagent 134 may be an analgesic or topical anesthetic, such as lidocaine. The device may include markings on the surface showing a user where to place a bodily fluid sample and at least one capillary structure to draw the bodily fluid sample into the absorptive matrix. The absorptive matrix may be pre-treated with a sugar, an excipient, a surfactant, an antimicrobial agent, an antifungal agent, a proteinase inhibitor, a DNase inhibitor, anticoagulant, or desiccant. The absorptive matrix 113 may include filter paper, cellulose, paper, or some other fibrous material. In some embodiments, the absorptive matrix 113 is a material that is soluble in aqueous solutions. Preferably, the absorptive matrix 113 has an open internal structure that promotes absorption of blood by capillary action and internal spaces dimensioned to receive extracellular vesicles 201 and hold the extracellular vesicles 110 intact.

In use, a person places the bodily fluid sample 201 on the indicated spot and allows the sample to dry. Instructions to that effect may be printed on the wrapper 105. The user may then ship the device 101 to a testing facility through a mail or parcel carrier.

The substrate 109 may be provided as a piece of cardstock or plastic. In some embodiments, the surface of the substate has at least (i) a first sample collection location that includes the absorptive matrix and (ii) a second sample collection location that includes a second absorptive matrix. The device 101 may be provided as part of a package that includes a lancet, a return envelope, and instructions directing the collection of multiple samples.

Any suitable fluid sample may be collected on the device 101. In preferred embodiments, the device 101 is used to collect a bodily fluid sample. The body fluid may preferably be saliva, urine, sweat, or blood. In an especially preferred embodiment, the body fluid is blood. Blood spot is obtained via a finger prick using a capillary or a lancet. The blood spot is collected on an absorptive matrix and allowed to dry. The absorptive matrix 113 may be cellulose, paper (e.g., filter paper), cotton gauze, fleece, polymer paper, or other absorptive material. The absorptive matrix may be pre-treated with one or any combination of a sugar, excipient, surfactant, antimicrobial agent, antifungal agents, a proteinase inhibitor, a DNase inhibitor, an anticoagulant, and a desiccant. The absorptive matrix 113 with the dried blood spot 103 can be mailed by the user to a laboratory or a testing facility for further processing and analysis.

The invention allows easy at-home sample collection and shipping for users without having to go to a testing facility to provide a sample. The invention is also useful for predicting if a person is susceptible to a disease, or to detect advancement or predict onset and/or reoccurrence. The invention can be utilized to detect any type of disease. In a preferred embodiment, the disease is cancer. For example, a person who has been diagnosed with or treated for cancer, is able to collect and dry a blood spot 103 on the absorptive matrix 113 at home, package, and ship the collection device 101 to a laboratory to detect or stage the cancer. Depending upon the disease stage, the physician is able to select a treatment plan for the person. Similarly, a person who has recovered from cancer can provide a dry blood spot in order to predict reoccurrence of the cancer.

Once absorptive matrix deposited with the dried blood spot is mailed to the testing facility, a sample can be prepared for further analysis without having to further process the sample. In one embodiment, the absorptive matrix is soluble in aqueous solutions used for eluting the extracellular vesicles 201 from the dried blood spot. One simple approach to extracting the EV 201 from the spot 103 is to punch a hole through the substrate 109, e.g., using a hole punch, thereby removing a disk of the absorptive matrix 113 (with an EV 201 therein) from the device 101. The disk of absorptive matrix 113 can be dropped into water in a suitable tube or container. Optionally, a size selection step (e.g., via chromatograph or similar) may be performed to specifically purify the solution for EVs 110. In one embodiment, the extracellular vesicles are eluted and isolated from the dry-blood spot using size exclusion chromatography. As mentioned above, the matrix can be constructed to extract and retain extracellular vesicles.

FIG. 2 shows EVs 110 in a solution 205 in a tube 207. Once the extracellular vesicles 110 are eluted into the aqueous solution 205, the extracellular vesicles are analyzed for biomarkers. In preferred embodiments, the extracellular vesicles are analyzed for the co-occurrence of the three or more biomarkers. In a preferred embodiment, co-occurrence of biomarkers are analyzed using magnetic beads 231 decorated with antibodies 235 against a first of the three biomarkers. Capture assay comprises the step of contacting the extracellular vesicles of interest with at least one capture agent. In a preferred embodiment, the capture agent may be a magnetic bead 231 with capture antibodies 235. The capture agent binds to at least one or more biomarkers present on the surface of the extracellular vesicle. Once captured, an assay is performed to detect one or disease specific biomarkers on the surface of the extracellular vesicles.

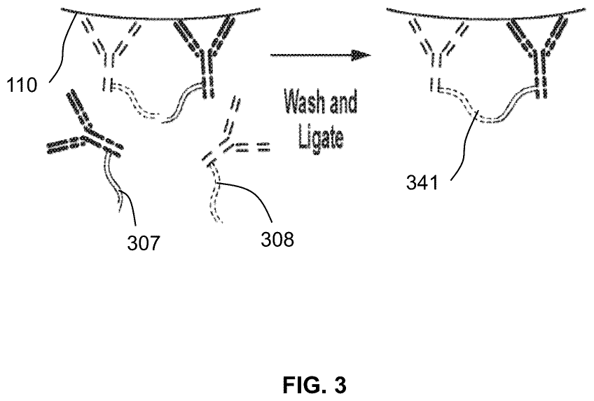

FIG. 3 illustrates steps of a proximity ligation assay that will produce a positive result if and only if the second and the third of the three biomarkers are present on the extracellular vesicle 110. The proximity ligation assay includes binding two antibodies that are linked to respective oligos 307, 308 to the second and third biomarkers, ligating the two oligos into a nucleic acid construct 341, and detecting the nucleic acid construct 341. As shown, the proximity ligation assays uses two antibody-DNA conjugates, although other ligands or reagents may be used. The DNA portions, the oligonucleotides 307, 308, of the two antibody-DNA conjugates are ligated together. The nucleic acid construct 341 maybe detected by a suitable reaction sush as quantitative PCR (qPCR).

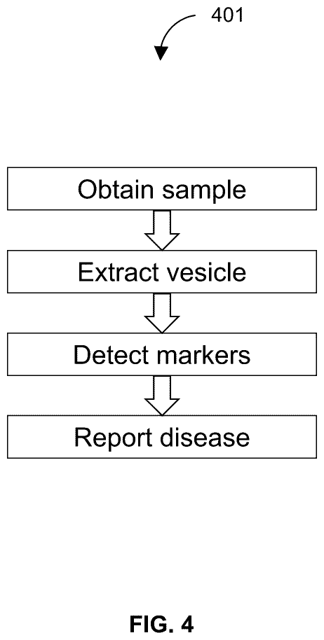

FIG. 4 shows steps of an analysis method 401 of the invention. The method 401 includes obtaining a sample collection device having a dried bodily fluid sample (e.g., blood) on a surface thereof, extracting at least one extracellular vesicle from the dried bodily fluid sample, detecting the co-occurrence of at least three biomarkers on the extracellular vesicle, and reporting the presence of a medical condition based on the detected co-occurrence of the three biomarkers. The fluid may have been obtained through a finger prick and/or using a capillary. The collection device may have an absorptive matrix on the surface, in which the bodily fluid sample is dried within the absorptive matrix. The absorptive matrix 113 may be pre-treated with a sugar, an excipient, a surfactant, an antimicrobial agent, an antifungal agent, a proteinase inhibitor, a DNase inhibitor, anticoagulant, a desiccant, or any combination thereof. The method 401 may include, prior to the obtaining step, sending the sample collection device to a person with instructions to place the bodily fluid sample on the surface and allow the bodily fluid sample to air-dry. The method 401 may include receiving the sample collection device 101 in the mail.

Preferably, the extracting step is carried out by eluting extracellular vesicles into an aqueous mixture. In some embodiments, the analyzing step comprises a binding assay to detect the three biomarkers. For example, a first biomarker may be captured with an antibody attached to magnetic bead, to purify the EVs from a solution. A second and third biomarker may be detected a proximity ligation assay. Preferably the three biomarkers are selected based on their co-occurrence in cells with the medical condition such as cancer.

Device and methods of the invention may use any combination (or “panel” or “biomarker signature”) for any specific disease such as, for lung cancer, those described in WO 2022/011197, incorporated by reference herein. For analysis of breast cancer, methods and devices of the invention may use markers disclosed in WO 2023/004078, incorporated by reference herein. For analysis of prostate cancer, methods and devices of the invention may use markers disclosed in WO 2023/004082, incorporated by reference herein. For analysis of pancreatic cancer, methods and devices of the invention may use markers disclosed in WO 2023/004080, incorporated by reference herein. For analysis of colorectal cancer, methods and devices of the invention may use markers disclosed in WO 2023/004077, incorporated by reference herein.

The invention also provides a test kit for at-home use. The test kit enables a user to obtain a fluid spot at home and then mail the kit to a testing facility for further analysis. Kits of the invention comprises a sterilizing agent for sterilization of the skin, a capillary or lancet for obtaining the blood spot through a finger prick, an absorptive matrix on which the blood spot can be collected as well as dried, and a shipping label to enable the user to mail the kit to a testing facility.

### Example 1

A collection kit comprising an absorptive surface, an antiseptic wipe, and a lancet is presented to a user. The user obtains a blood droplet with the lancet after applying the antiseptic and deposits the blood spot onto the absorptive surface, here a filter paper. The filter paper is pre-treated with substances that preserve extracellular vesicles. The blood spot is air-dried by exposing the filter paper to air for a period of time per included instructions. The dried blood spot deposited on the filter paper is mailed to a testing facility. Upon receiving the dried blood spot on the filter paper, the filter paper was added to a tube comprising phosphate buffer solution (PBS) for 1 hour. The eluate is run through size-exclusion chromatography (SEC) column to isolate target extracellular vesicles based on their size. The extracellular vesicles are analyzed for presence of lung adenocarcinoma biomarkers. The presence of lung adenocarcinoma is reported when sTn, MUC1, and CEACAM6 biomarkers are detected together on an EV by methods of the disclosure.

### Example 2

Whole blood was collected via finger prick and deposited on Whatman #2 and Whatman #3 filter paper. Samples were either eluted as provided or spiked with COV413A extracellular vesicles (obtained from a cancer cell line). The blood spots were dried and eluted from the filter paper. Two combinations were used, one for that looked at three tetraspanins (CD63, CD81, CD9) which are ubiquitously expressed on extracellular vesicles. The second combination was an ovarian cancer combination MUC1, BST2 +FOLR1), which only gives a strong signal when the blood is spiked with COV413A extracellular vesicles. The signal is greatly diminished when detergent is added, suggesting that much of the signal is derived from the extracellular vesicles. One of the samples was run through a size-exclusion chromatography (SEC) column after it was eluted from the filter paper. This was done to ensure that no cell fragments were in the sample that might lead to misleading signal. There is strong signal, even from the SEC-treated sample. The detergent data and data from the samples suggests that extracellular vesicles form a large part of the signal obtained from dried blood spots.

The data presented were analyzed and the Ct of each group was subtracted from the blank sample (Whatman #2 paper with PBS) to yield the ΔCt. The larger the ΔCt, the stronger the signal. The results show that the signals in blood without detergent are larger than that of blood with detergent. Lastly, COV413A EVs were spiked, in PBS, onto the paper. The signal is weaker than expected. It's possible the difference is that extracellular vesicles in blood are protected by serum proteins and are better preserved than extracellular vesicles spiked into buffer.

What is claimed is:

1. A method for evaluating disease status, comprising the steps of:
obtaining a sample collection device having a dried bodily fluid sample on a surface thereof;
extracting at least one extracellular vesicle from the dried bodily fluid sample;
detecting presence of a biomarker on the extracellular vesicle; and
evaluating disease status based on the presence of the biomarker.

2. The method of claim 1, further comprising sending the sample device to an individual for sample collection.

3. The method of claim 1, wherein the detecting step comprises detecting a plurality of extracellular vesicle-associated biomarkers.

4. The method of claim 3, wherein the plurality comprises three biomarkers that occur on a single extracellular vesicle.

5. The method of claim 1, wherein the bodily fluid is blood.

6. The method of claim 1, wherein the surface comprises an absorptive matrix.

7. The method of claim 6, wherein the matrix is pre-treated with a sugar, an excipient, a surfactant, an antimicrobial agent, an antifungal agent, a proteinase inhibitor, a DNase inhibitor, or a desiccant.

8. The method of claim 1, wherein the extracting step is carried out by eluting extracellular vesicles into an aqueous mixture.

9. The method of claim 1, wherein the analyzing step comprises a binding assay to detect the biomarker.

10. The method of claim 4, wherein a first biomarker is bound to a magnetic bead by an antibody, and a second and third biomarker are detected a proximity ligation assay.

11. The method of claim 10, wherein the proximity ligation assay includes binding two oligos to the second and third biomarkers, ligating the two oligos into a nucleic acid construct, and detecting the nucleic acid construct.

12. The method of claim 10, wherein the magnetic bead is used to hold the extracellular vesicle during a wash step.

13. The method of claim 11, wherein the proximity ligation assays uses two antibody-DNA conjugates.

14. The method of claim 10, wherein the DNA portion of the two antibody-DNA conjugates are ligated together.

15. The method of claim 4, wherein the three biomarkers are selected based on their co-occurrence in cells with the medical condition.
